(19) United States
(12) Patent Application Publication
(10) Pub. No.: US 2006/0147408 A1
(43) Pub. Date: Jul. 6, 2006

(54) Skin cleansing and sloughing solution

(75) Inventor: Vickey A. Reitz, Tulsa, OK (US)

Correspondence Address: WINSTEAD SECHREST & MINICK P.C., PO BOX 50784, DALLAS, TX, 75201, US

(73) Assignee: VISION ENDURANCE RESOURCE, INC., Tulsa, OK (US)

(21) Appl. No.: 11/030,429

(22) Filed: Jan. 6, 2005

(51) Int. Cl. A61K 8/97; A61K 36/61

(52) U.S. Cl. 424/74; 424/769

(57) ABSTRACT

A composition and process for removing dry and callused skin from a human foot comprising the steps of: a) providing a composition consisting essentially of at least about 37.2% (w/v) but less than 54.31% (w/v) isopropyl alcohol; about 29.59% (w/v) but less than 73.97% (w/v) sodium bicarbonate and about 0% (w/v) to about 0.63% (w/v) Melaleuca alternifolia oil; b) applying the composition of (a) to the interior bottom portion of a shoe having contiguous front, rear, side and lower and upper portions dimensioned and configured to substantially surround a human foot when inserted therein; c) introducing the human foot to the composition of (a) as applied in (b); and d) removing the human foot as introduced in (c) to the composition of (a) at a time subsequent to the introduction.

### REFERENCE TO PENDING APPLICATIONS

This application is not related to any pending application.

### REFERENCE TO MICROFICHE APPENDIX

This application is not referenced to any microfiche appendix.

### FIELD OF INVENTION

This invention relates to a composition in method for removing dry and callused skin from a human foot or other appendage. The invention is useful in those instances where surgical removal of calluses or repeated applications of skin sloughing solution are deemed impractical.

### BACKGROUND OF INVENTION

Typically, cramming a foot into a shoe for a period of 12 to 16 hours a day subjects the skin of the foot to repeated friction and pressure. Skin often protects itself by building up callused portions in response to this friction/pressure. Calluses are readily identified as thick, hardened, (usually) harmless, and sometimes dry layers of skin.

Corns and calluses are areas of thick skin that result form excessive pressure or friction over a boney prominence. When these areas develop on the bottom of the foot they are called calluses. When they occur on the top of the toes they are called corns. They can also occur between the toes, the back of the heels and the top of the foot. The thickening of the skin is a normal body response to pressure or friction. Often times they are associated with a projection of bone called a bone spur.

The most common area for the formation of calluses on the bottom of the foot is in the area of the ball of the foot. This is a weight bearing area where the long bones behind the toes, (metatarsals), bear the greatest amount of weight and pressure. If one or more of these metatarsals is out of alignment, excessive pressure is generated in the area producing a callous. The callused area can be very discreet and have a “core” or they can be more dispersed covering a larger area. These areas can become quite painful as the skin thickens. People who have diabetes are at risk of these areas breaking down and producing sores or ulcerations that can become infected and are often warned not to try home remedies to alleviate these maladies.

There are numerous over the counter treatments for corns and calluses. Some of these remedies contain an acid that burn off the callous. Care must be taken when using these medications as if used incorrectly they can cause a chemical burn to the skin. Additionally, these remedies are of temporary nature as because the source of the pressure has not been alleviated. Professional treatment of calluses typically consists of using a special shoe insert called a functional orthotic that corrects foot function. In certain instances, surgery may be required and is typically directed at correcting the alignment of the offending bone. Cutting out the callous by lay persons is dangerous and will likely make the callusing condition worse if the underling boney problem is not corrected.

Corns are areas of thick skin that most commonly occur on the top of the toes. Small corns can also occur on the side of the little toe next to the toenail. A small bone spur usually causes this problem. Bone spurs also cause corns between the toes. Soft corns are areas of white moist skin between the toes and most commonly occur between the fourth and fifth toes. These corns can be very painful, and if not treated can form small ulcerations that can become infected.

Home treatment of corns is often directed at reducing the pressure between the toes with cotton or a foam cushion and using an antibiotic ointment to reduce the risk of infection. Professionals often counsel against using over the counter corn removers in this area because of the risk of increased damage to the skin resulting in infection. Professional treatment may consist of removing the irregular shaped bone that causes the development of the corn. Quite often, patients prefer that the doctor simply trim down and pad the callused areas.

It is therefore seen that there still remains a need for a process and composition for removing dry and callused skin which avoids the hazards and repeated application of contemporary art solutions and practices.

### SUMMARY OF INVENTION

It is therefore an object of the present invention to provide a process and composition for removing dry and callused skin from an human appendage, such as a foot, via non-invasive practice.

It is another object of the instant invention to provide a skin sloughing solution in which Melaleuca alternifolia (tea tree) oil is combined with isopropyl alcohol and sodium bicarbonate to effectuate the sloughing of dry or callused skin.

It is another object of the present invention to provide a skin sloughing composition with improved cleansing characteristics.

It is yet a further object of the present invention to provide an improved skin sloughing solution which eliminates the nicks and cuts inherent with the excising of callused skin by razors, knives and other similarly intended utensils.

An embodiment of the present invention is a composition and process for removing dry and callused skin from a human foot comprising the steps of: a) providing a composition consisting essentially of at least about 37.2% (w/v) but less than 54.31% (w/v) isopropyl alcohol; about 29.59% (w/v) but less than 73.97% (w/v) sodium bicarbonate and about 0% (w/v) to about 0.63% (w/v) Melaleuca alternifolia oil; b) applying the composition of (a) to the interior bottom portion of a shoe having contiguous front, rear, side and lower and upper portions dimensioned and configured to substantially surround a human foot when inserted therein; c) introducing the human foot to the composition of (a) as applied in (b); and d) removing the human foot as introduced in (c) to the composition of (a) at a time subsequent to the introduction.

### DETAILED DESCRIPTION OF THE INVENTION

While the making and using of various embodiments of the present invention are discussed in detail below, it should be appreciated that the present invention provides for inventive concepts capable of being embodied in a variety of specific contexts. The specific embodiments discussed herein are merely illustrative of specific manners in which to make and use the invention and are not to be interpreted as limiting the scope of the instant invention.

The claims and specification describe the invention presented and the terms that are employed in the claims draw their meaning from the use of such terms in the specification. The same terms employed in the prior art may be broader in meaning than specifically employed herein. Whenever there is a question between the broader definition of such terms used in the prior art and the more specific use of the terms herein, the more specific meaning is meant.

To prepare the composition of the invention, at least about 37.2%, but less than 54.31% (w/v) isopropyl alcohol is combined with about 29.59%, but less than 73.97% (w/v) sodium bicarbonate and 0% to about 0.63% (w/v) Melaleuca alternifolia oil under slight agitation with a minimal amount of air entrapment to form a solution mixture. The solution mixture is prepared under agitation for a period sufficient to blend the ingredients whereupon the solution is ready for immediate use. An example of a period sufficient to blend the ingredients is 1-2 minutes. The composition can be manufactured with air entrained or entrapped in it, and an effective product will yet result. Though it is noted a resulting product with air entrapped will be somewhat more opaque and less viscous than a resulting product absent entrapped air.

The following table provides detailed disclosure with respect to best mode and preferred composition as follows:

TABLE 1

| Percentage Weight by Volume | Solution |
|---|---|
| isopropyl alcohol | 48.61% |
| sodium bicarbonate | 59.17% |
| Melaleuca alternifolia oil | 0.42% |

Once prepared, the composition is freely applied to the interior bottom portion of a shoe having a continuous and attached front, rear, side and lower and upper portions dimension and configured to substantially surround a human foot when inserted therein. Examples of such footwear are commonly referred to as “dress shoes” by that portion of the public wearing such articles. The dry and callused portion of the human foot is introduced to the composition previously introduced to the interior bottom of a shoe as described above and the shoe worn continuously for a period of time. An example of this period of time is six to eight hours. At the conclusion of the afore-noted period, the foot is removed from the shoe and dry/callused skin is removed by washing or otherwise frictionally engaging the now loosened callused/dry skin with a washcloth or other similarly intended article. The solution following practice adopts a powdery consistency and may be merely shaken or brushed from, shoes, sock, hosiery, or other clothing articles previously exposed to the solution of the present invention.

Although the invention has been described with reference to specific embodiments, these descriptions are not meant to be construed in a limiting sense. Various modifications of the disclosed embodiments, as well as alternative embodiments of the invention will become apparent to persons skilled in the art upon reference to the description of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

It is therefore, contemplated that the claims will cover any such modifications or embodiments that fall within the true scope of the invention.

What is claimed is:

1. A process for removing dry and callused skin from a human foot comprising the steps of:
a) providing a composition consisting essentially of at least about 37.2% (w/v) but less than 54.31% (w/v) isopropyl alcohol; about 29.59% (w/v) but less than 73.97% (w/v) sodium bicarbonate and about 0% (w/v) to about 0.63% (w/v) Melaleuca alternifolia oil;
b) applying the composition of (a) to the interior bottom portion of a shoe having contiguous front, rear, side and lower and upper portions dimensioned and configured to substantially surround a human foot when inserted therein;
c) introducing the human foot to the composition of (a) as applied in (b); and
d) removing the human foot as introduced in (c) to the composition of (a) at a time subsequent to the introduction.

2. The process of claim 1 further comprising immersing the human foot previously introduced to the composition of (a) in a solution of water and the composition of (a).

3. The process of claim 2 further comprising brushing and towel drying the previously immersed portions of the human foot in a manner to slough or otherwise remove loose skin.

4. The process of claim 1 wherein the isopropyl alcohol in the composition is in the range of 42.91% (w/v) to 51.46% (w/v) by weight, the sodium bicarbonate is in the range of 44.38% (w/v) to 66.57% (w/v) and the Melaleuca alternifolia oil in the range of 0.21% (w/v) to 0.53% (w/v).

5. The process of claim 4 wherein the isopropyl alcohol in the composition is 48.61% (w/v), the sodium bicarbonate is 59.17% (w/v) and the Melaleuca alternifolia oil in the 0.42% (w/v).

6. A composition to remove dry and callused skin from a human foot comprising essentially of at least about 37.2% (w/v), but less than 54.31% (w/v) isopropyl alcohol; about 29.59% (w/v), but less than 73.97% (w/v) sodium bicarbonate and about 0% (w/v) to about 0.63% (w/v) of Melaleuca alternifolia oil.

7. The composition of claim 6 wherein the isopropyl alcohol in the composition is in the range of 42.91% (w/v) to 51.46% (w/v) by weight and the sodium bicarbonate is in the range of 44.38% (w/v) to 66.57% (w/v) and the Melaleuca alternifolia oil is in the range of 0.21% to 0.53% (w/v).

8. The composition of claim 7 wherein the isopropyl alcohol in the composition is 48.61% (w/v), the sodium bicarbonate is 59.17% (w/v) and the Melaleuca alternifolia oil is 0.42% (w/v).
